(19) United States
(12) Patent Application Publication
(10) Pub. No.: US 2010/0184732 A1
(43) Pub. Date: Jul. 22, 2010

(54) METHOD OF LONG-TERM TREATMENT OF GRAFT-VERSUS-HOST DISEASE USING TOPICAL ACTIVE CORTICOSTEROIDS

(76) Inventor: George McDonald, Bellevue, WA (US)

Correspondence Address: Lewis Kohn & Fitzwilliam LLP, 10935 Vista Sorrento Parkway, Suite 370, San Diego, CA, 92130, US

(21) Appl. No.: 12/621,436

(22) Filed: Nov. 18, 2009

Related U.S. Application Data
(63) Continuation-in-part of application No. 10/613,788, filed on Jul. 3, 2003, now abandoned.
Application No. 10/613,788 is a continuation of application No. 09/753,814, filed on Jan. 3, 2001, now abandoned.
(60) Provisional application No. 60/233,194, filed on Sep. 15, 2000.

(51) Int. Cl. A61K 31/573; A61K 31/57

(52) U.S. Cl. 514/170; 514/180

(57) ABSTRACT

A method of long-term therapy using corticosteroids to treat tissue damage associated with graft-versus-host disease in a patient having undergone hematopoietic cell transplantation, and host-versus-graft disease in a patient having undergone organ allograft transplantation. The method includes orally administering to the patient a therapeutically effective amount of a topically active corticosteroids, such as beclomethasone dipropionate, from the 29^{th} day until the 56^{th} day following hematopoietic cell or organ allograft transplantation. Representative tissues include tissue of the intestine and liver, while representative tissue damage includes inflammation thereof.

### RELATED APPLICATIONS

This application is a Continuation-In-Part of U.S. patent application Ser. No. 10/613,788 filed on Jul. 3, 2003 which is a Continuation of U.S. patent application Ser. No. 09/753,804 filed on Jan. 3, 2001, now abandoned, which claims the benefit of priority from U.S. Provisional Patent Application No. 60/233,194 filed on Sep. 15, 2000, the entire contents of which are herein incorporated by reference.

### FIELD OF THE INVENTION

This invention relates to the long-term treatment of Graft-Versus-Host Disease (GVHD) and more, particularly to the treatment of intestinal or gastrointestinal Graft-Versus-Host Disease by an orally effective therapeutic agent.

### BACKGROUND OF THE INVENTION

Graft-versus-host disease (GVHD) is a complication of allogeneic hematopoietic cell transplantation in which tissues of the host, most frequently the skin, liver and intestine, are damaged by lymphocytes from the donor. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipients of HLA-mismatched marrow. Patients with mild intestinal GVHD present symptoms of anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition.

Standard initial treatment for acute GVHD includes systemic immunosuppressive agents, usually high-dose prednisone at 2 mg per kg per day added to prophylactic medications such as methotrexate, cyclosporine and tacrolimus. Prednisone achieves a complete and sustained remission of gastrointestinal symptoms in 50-70% of patients with GVHD. Patients who fail to respond receive therapy with additional immunosuppressive regimens, such as higher-dose prednisone, anti-thymocyte globulin, and investigational anti-T-cell monoclonal antibodies or immunotoxins. Unfortunately, the risks of prolonged immunosuppressive therapy are significant, especially among patients with immature marrow grafts. These risks include local and disseminated infection, the development of lympho-proliferative disease, and systemic glucocorticoid side effects such as hypothalamic-pituitary-adrenal axis suppression, myopathy, neuropsychiatric disease, and bone demineralization.

Recently, investigators have reported the results of a phase I trial of topically active corticosteroid, beclomethasone dipropionate (BDP), for the treatment of patients with intestinal GVHD (Baehr et al., Transplantation 60:1231-1238, 1995). In this trial, BDP capsules were given orally, 8 mg daily, half as enteric-coated capsules designed to dissolve in the alkaline pH of the upper small intestine, and half of the capsules that dissolve in the stomach. Significant improvement was found in the appetite, oral intake, nausea, and diarrhea over the course of therapy with oral BDP alone and with oral BDP added to prednisone therapy. However, the time to improvement in patients receiving BDP as monotherapy was 7-10 days, which is longer than the response usually seen with prednisone therapy.

A drawback with the above regimen is that treatment is initiated with BDP only after presentation of symptoms of intestinal GVHD, with typical patient enrollment at a mean of 58 days post-transplant (i.e., ranging from day 21-231 after transplant). The difficulty with treatment after presentation of intestinal GVHD symptoms is that significant inflammation and/or damage to the intestine has already occurred prior to initiation of therapy. Severe damage to the lining of the intestine is often fatal, as malnutrition, protein loss, and blood stream infections preclude regeneration of lining cells. This study did, however, provide evidence that oral BDP therapy was safe and effective in the treatment of mild-to-moderate intestinal GVHD, taken alone or when added to prednisone.

A related condition to GVHD is host-versus-graft disease (HVGD), also referred to as organ allograft rejection. HVGD disease may occur, for example, when a donor intestine is transplanted into a patient with a diseased intestine. In this case, cells of the patient's immune system (the host) may attack the foreign intestinal tissue (the graft). While intestinal transplantation is not routine at the present time, such techniques will likely become more common. Thus, prophylactic medications are needed to prevent HVGD for many of the reasons noted above with regard to GVHD.

While significant advances have been made with regard to the treatment of GVHD following bone marrow transplantation, there is still a need in the art for improved methods, particularly in the context of preventing the intestinal mucosal damage associated with the onset of GVHD. Such preventative methods should begin immediately following hematopoietic cell transplantation, and reduce tissue damage associated with the subsequent onset of GVHD. There is also a need for methods to prevent HVGD in the context of, for example, intestinal or liver transplantation. The present invention fulfills these needs and provides further related advantages.

### SUMMARY OF THE INVENTION

In brief, this invention discloses a method for long term treatment of tissue damage, particularly of the intestinal and/or liver, caused by graft-versus-host disease (GVHD) that commonly follows hematopoietic cell transplantation, or caused by host-versus-graft disease (HVGD) or organ allograft rejection.

Hematopoietic cell transplantation is the generic term that encompasses bone marrow transplantation, peripheral blood stem cell transplantation, umbilical vein blood transplantation, or any other source of pleuripotent hematopoietic stem cells. The method includes the oral administration of an effective amount of a topically active corticosteroid (abbreviated herein as “TAC”) to a patient having undergone hematopoietic cell transplantation. A representative TAC of this invention is beclomethasone dipropionate (BDP). Such long term administration starts following the hematopoietic cell transplantation and continues up to day 56 following the hematopoietic cell transplantation, thereby treating, delaying and/or reducing severity of the symptoms-normally associated with tissue damage caused by GVHD.

As mentioned above, this invention is directed to a method for long term treatment of tissue damage caused by graft-versus-host disease (GVHD) which commonly follows hematopoietic cell transplantation, as well as by host-versus-graft disease (HVGD) or allograft rejection which commonly follows organ transplantation. The method of the present invention employs oral administration of a long term treatment effective amount of a topically active corticosteroid (TAG) to a patient having undergone hematopoietic cell or organ allograft transplantation.

Representative TACs include, but are not limited to, beclomethasone dipropionate, alciometasone dipropionate, busedonide, 22S busesonide, 22R budesonide, beclomethasone-17-monopropionate, clobetasol propionate, diflorasone diacetate, flunisolide, flurandrenolide, fluticasone propionate, halobetasol propionate, halcinocide, mometasone furoate, and triamcinalone acetonide. Such TACs are well known to those skilled in the field of, for example, intestinal disorders, and are commercially available from any number of sources. Suitable TACs of this invention have rapid first-pass metabolism in the intestine and liver, low systemic bioavailability, high topical activity, and rapid excretion (see, e.g., Thiesen et al., Alimentary Pharmacology & Therapeutics 10:487-496, 1996) (incorporated herein by reference).

These and other aspects of this invention will be evident upon reference to the following detailed description.

### BRIEF DESCRIPTION OF THE FIGURES

FIG. 1: Cumulative treatment failures during the first 50 days in patients receiving placebo or BDP.

FIG. 2: the cumulative treatment failures during the first 80 days in patients receiving placebo or BDP.

FIG. 3: Duration of overall survival—post-randomization (safety population)

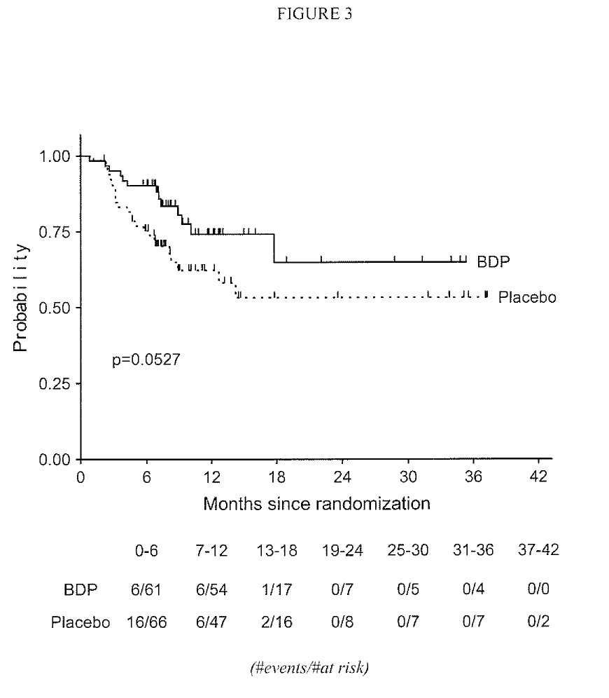

FIG. 4: Prednisone and Study Drug Dosing

### DETAILED DESCRIPTION OF THE INVENTION

### Definitions

As used above and throughout the instant specification and appending claims, the following terms, unless otherwise indicated, shall be understood to have the following meanings:

As used herein, the term “long-term treatment” means administration of an effective therapy to reduce the severity of the symptoms associated with GVHD with administration beginning on day 29 following hematopoietic cell transplantation and continuing at least until day 56 or HVGD following organ allograft transplantation. Long-term treatment may continue for several dosing cycles of 29 days administered consecutively until the desired therapeutic effect is produced in the patient. In the alternative, the term “long-term treatment” means administration of an effective therapy to reduce the severity of the symptoms associated with GVHD with administration beginning immediately following hematopoietic cell transplantation and continuing through, and potentially beyond, day 56 following hematopoietic cell transplantation.

As used herein, the term “tissue” means intestinal mucosa or the small bile ducts in the liver. Intestinal mucosa includes mucosa of the esophagus, stomach, small intestine and colon.

As used herein, the term “damage” means a range of symptoms from mild inflammation to destruction of the mucosa of the intestine to fatal exfoliation of intestinal epithelial cells. Inflammation typically presents as fever, abdominal pain, nausea, vomiting, diarrhea, intestinal bleeding, and jaundice.

As used herein, the term “patient” means a human or other mammal.

As used herein, the term “effective amount” is meant to describe an amount of a compound effective in producing the desired therapeutic effect.

### Preferred Embodiments

In one embodiment, the tissue damage is caused by intestinal inflammation associated with intestinal graft-versus-host disease in a patient having undergone hematopoietic cell transplantation. In this embodiment, an effective amount of a TAC is orally administered to a patient in need thereof from day 29 to day 56 following hematopoietic cell transplantation.

In another embodiment, an effective amount of a TAC is orally administered to a patient in need thereof immediately following hematopoietic cell transplantation and continuing beyond day 56 following hematopoietic cell transplantation.

In another embodiment, the tissue damage is caused by HVGD or organ allograft rejection, including (but not limited to) intestinal or liver transplantation. In this embodiment, an effective amount of a TAO is orally administered to a patient in need thereof from day 29 to day 56 following hematopoietic cell transplantation.

In more specific embodiments, the TAO is administered orally at a dosage of 4 mg/day to 12 mg/day in a form suitable for oral administration, such as capsules, pills, coated microspheres with specific dissolution qualities, or emulsions. Other agents may optionally also be included in such oral formulations.

In one embodiment of this invention, the TAC is beclomethasone dipropionate (BDP). BDP is a compound which is available from a number of commercial sources, such as Schering-Plough Corporation (Kenilworth, N.J.) in bulk crystalline form, and has the following structure (i.e., beclomethasone 17,21-dipropionate):

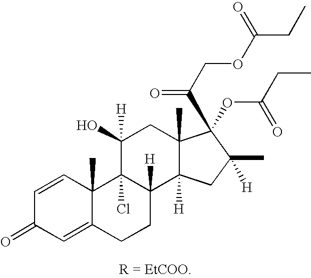

In another embodiment, the TAC is administered to patients having undergone hematopoietic cell or organ allograft transplantation, i.e. allogenic hematopoietic cell recipients who have typically received marrow-ablative chemotherapy and/or total body irradiation followed by donor hematopoietic cell infusion, or patients having undergone intestinal or liver transplantation. Such procedures have been widely disclosed, and are well known to those skilled in this field.

Transplant patients receive a therapeutically acceptable amount of a TAC by oral administration. The TAC may be formulated for oral administration by techniques well known in the formulation field, including formulation as a capsule, pill, coated microsphere with specific dissolution qualities, or emulsion. Suitable capsules or pills generally contain from 1 mg to 2 mg TAC, and typically about 1 mg TAC, plus optional fillers, such as lactose, and may be coated with a variety of materials, such as cellulose acetate phthalate. By appropriate coating, such capsules, microspheres or pills may be made to dissolve within various location of the intestinal tract. For example, enteric-coated capsules prepared with a coating of cellulose acetate phthalate are known to dissolve in the alkaline environment of the small bowel, thus delivering its content to the small bowl and colon. Emulsions containing a TAC may also be employed for oral delivery, including optional emulsifying agents.

In addition to the TAC, acceptable carriers and/or diluents may be employed and are familiar to those skilled in the art. Formulations in the form of pills, capsules, microspheres, granules or tablets may contain, in addition to one or more TACs, dituents, dispersing and surface active agents, binders and lubricants. One skilled in the art may further formulate the TAC in an appropriate manner, and in accordance with accepted practices, such as those disclosed in Remington's Pharmaceutical Sciences, Gennaro, Ed., Mack Publishing Co., Easton, Pa., 1990 (incorporated herein by reference).

In the practice of this invention, a “long term therapeutically effective amount” of a TAO is administered to a patient in need thereof. In general terms, a long term therapeutically effective amount of a TAC is an amount which, when delivered orally, beginning on day 29 and continuing to day 56 following hematopoietic cell transplantation, or associated with HVGD following organ allograft transplantation. Such an amount may be readily determined by one skilled in the art by well known dose-response investigations, and will generally range from 4 mg/day to 12 mg/day, and more typically range from 6 mg/day to 8 mg/day.

As optional components, other active long-term agents may be administered in combination with the TAC, including (but not limited to) prednisone, prednisolorie, cyclosporine, methotrexate, tacrolimus and biological agents that affect T-lymphocytes.

In the context of GVHD, long term therapeutic administration of a TAC beings 29 days after infusion of hematopoietic cells, and continues until 56 days after infusion of hematopoietic cells. An important aspect of this invention is that the TAC is orally administered such that it is topically administered to the intestinal and/or liver tissue. Thus, oral administration, as that term is used herein, is not intended to encompass systemic administration, such as by intravenous injection. Rather, the TAC has little (if any) systemic availability, but high topical activity on intestinal and/or liver tissue. Such limited distribution results in fewer side effects, which is a significant advantage of this invention.

In addition to differences with regard to location and timing of administration, there is also a biological basis between short-term and long term treatment regiments. Corticosteroids have been used in the acute or short term management of GVHD. Conversely, long term therapy using corticosteroids is not appropriate use to the systemic side effects of compounds, such as prednisone. Therefore, using BDP for long term therapy, it is able to control the symptoms of GVHD without having systemic exposure to steroid toxicity.

In treatment, the objectives are to suppress a wide variety of biological events that have already resulted in tissue destruction, for example, the generation of inflammatory cytokines, the recruitment of additional inflammatory cells to the site of injury, the destruction of the barrier function of the intestinal mucosa (the lining), the passage of bacteria and toxins through the damaged intestinal mucosa, the up-regulation of biologic responses to bacteria and endotoxin, and the widespread organ responses to these events (such as leaky blood vessels, increased cardiac output, decreased systemic vascular resistance, diffuse lung injury, and renal insufficiency). When a patient has GVHD, treatment is successful only 50-75% of the time; the remainder of the patients generally dies.

By appropriate formulation of the TAC (such as enterically coated capsules), it can be delivered to the entire mucosal surface of the intestine in high doses. Thus, TAC can achieve high concentrations in the intestinal mucosa where this initiating alloimmune recognition event is taking place. It is believed that blunting the initiating event prevents the large cascade of biologic events that make up the syndromes of GVHD and HVGD.

It will be appreciated that, although specific embodiments of this invention have been described herein for purpose of illustration, various modifications may be made without departing from the spirit and scope of the invention.

### Clinical Studies with Oral BDP for the Treatment of GVHD

Oral BDP has been studied in patients with gastrointestinal manifestations of GVHD at Fred Hutchinson Cancer Research Center (FHCRC) for several years. Two studies were conducted under an investigator Investigational New Drug application (IND) in the 1990s to study the safety and efficacy of oral BDP in patients with GVHD with primarily gastrointestinal symptoms. These two studies are listed in Table 1, and each is summarized below.

TABLE 1
Clinical Studies with Oral BDP

| | Study Title and | | | | BDP Dosage |
|---|---|---|---|---|---|
| Section | Reference | Design | Patients | N | Regimen |
| 5.2.2.1 | Oral Beclomethasone | Phase ½ | gastrointestinal GVHD | 23 | 8 mg/day [two 1 mg |
| | Dipropionate for the | open-label | Patients receiving only | | capsules (one IR |
| | Treatment of Patients | uncontrolled | prophylactic | | and one ER) q.i.d.] |
| | with Gastrointestinal | | immunosuppressive | | for 28 days |
| | Graft-Versus-Host | | therapy | | |
| | Disease (Study No. | | gastrointestinal GVHD | 17 | |
| | 615) (Baehr et al. | | patients receiving | | |
| | 1995) | | prednisone (0.25-4 mg/kg) | | |
| 5.2.2.2 | Controlled Study of | Phase 2 | gastrointestinal GVHD | 31 | 8 mg/day BDP [two |
| | Prednisone With or | stratified, | II patients receiving 1 mg/kg/ | | 1 mg capsules (one |
| | Without Oral | randomized, | day of | | IR and one EC) |
| | Beclomethasone | double-blind, | prednisone for 10 days | | q.i.d.] for 30 days |
| | Dipropionate for the | placebo- | (dose was rapidly | 29 | placebo q.i.d. for 30 |
| | Initial Treatment of | controlled | tapered starting on day | | days |
| | Patients with | | 11 to a low dose of | | |
| | Intestinal Graft- | | 0.125 mg/kg/day by day | | |
| | Versus-Host Disease | | 17) | | |
| | (Study No. 875) | | | | |
| | (McDonald et al. | | | | |
| | 1998) | | | | |

q.i.d. = four times daily

### 5.2.2.1 Oral Beclomethasone Dipropionate for the Treatment of Patients with Gastrointestinal Graft-Versus-Host Disease (Study No. 615)

This study was an uncontrolled pilot study in 40 GVHD patients, 17 of whom were already taking prednisone for their GVHD at the time of enrollment, and 23 patients who were not (Baehr et al. 1995). Thus, the former group was not responding well to systemic corticosteroids, and the question was whether they would benefit from the addition of a potent, locally acting, corticosteroid administered orally. The question in the latter group was whether such a drug by itself would be adequate to control the gastrointestinal symptoms of GVHD. Both groups of patients received 8 mg/day of oral BDP (2 mg q.i.d.) for 28 days. Each 2 mg dose consisted of one EC (1 mg) and one IR (1 mg) capsule of BDP.

Using the baseline status of patients as their own controls, up to 28 days of BDP resulted in the abatement of most gastrointestinal symptoms and an increase in oral food intake in both groups of patients (i.e. taking prednisone or not). The timing of response in the BDP alone group appeared to be slower than historical experience with similar patients treated with high dose prednisone.

No significant treatment-related adverse events were observed in this study. Study drug associated adverse events reported during the trial included mild metallic taste and mild peri-umbilical heaviness. In the patients not receiving prednisone, tests for HPA axis suppression showed evidence of suppression occurred in the majority of patients. Despite the evidence of some suppression, all of these patients were able to respond at least partially to ACTH administration with an increase in serum cortisol levels. None of these patients developed clinical symptoms of hypocortisolism.

### 5.2.2.2 Controlled Study of Prednisone with or without Oral Beclomethasone Dipropionate for the Initial Treatment of Patients with Intestinal Graft-Versus-Host Disease (Study No. 875)

This study was a placebo-controlled phase 2 trial conducted in 60 patients with Grade II GVHD with gastrointestinal symptoms (Study No. 875) (McDonald et al. 1998). In this study, oral BDP was studied as an adjunctive therapy to 10 days of prednisone, i.e. all patients received prednisone and were also randomized to receive active or placebo BDP capsules for 30 days. Oral BDP was administered at 8 mg/day of oral BDP (2 mg q.i.d.). Each 2 mg dose consisted of one EC (1 mg) and one IR (1 mg) capsule of BDP. After day 10, the dose of prednisone was rapidly tapered to a dose of 0.125 mg/kg/day. The primary efficacy endpoint for this study was the proportion of patients in each of the groups who were eating ≧70% of their expected caloric requirements (ECR).

Treatment with oral BDP as an adjuvant to prednisone resulted in a significantly higher proportion of patients with increased oral caloric intake compared to treatment with prednisone plus placebo. Gastrointestinal symptoms (nausea, vomiting, diarrhea) all improved in the BDP group compared to the placebo group. BDP was well tolerated, and the incidence of infections between the BDP and placebo groups was similar.

### EXAMPLES

A subsequent study was undertaken to assess the effect of a longer term treatment of patients with grade II Graft vs. Host Disease with gastrointestinal symptoms regimen with orally administered BDP in conjunction with ten days of high-dose prednisone therapy. The primary objective of this multi-center study was to compare the efficacy, defined as time to treatment failure, of an oral BDP regimen (1 mg/kg/day prednisone for 10 days plus 2 mg oral BDP q.i.d. for 50 days) with the efficacy of standard of care (1 mg/kg/day of oral prednisone administered for 10 days plus matching placebo tablets for 50 days) in patients with Grade II graft versus host disease (GVHD) with gastrointestinal (GI) symptoms. The Secondary objectives of the study were:
1. To compare the proportion of treatment failures in the two groups on Study Days 10, 30, 50, 60, and 80.
2. To compare the treatment groups with respect to cumulative systemic corticosteroid exposure.
3. To compare the treatment groups with respect to the incidence and degree of hypothalamic-pituitary-adrenal (HPA) axis suppression in patients who have not experienced treatment failure by Study Day 50.
4. To evaluate the safety of BDP by comparing the treatment groups with respect to treatment-emergent adverse events.
5. To compare the treatment groups with respect to total deaths and cause of death through 200 days post-transplant.
6. To investigate the pharmacokinetic (PK) profile of single and multiple dose administration of 2 mg oral BDP four times daily (split evenly between the immediate release [IR] and the enteric coated [EC] tablets) for 50 days in patients with Grade II GVHD with GI symptoms.

To be eligible for entry into the study, patients must be at least 10 days post allogeneic hematopoietic cell transplantation, have GI symptoms consistent with Grade II GVHD, and have endoscopic evidence of GVHD. The diagnosis of GVHD was confirmed by biopsy of the intestine (esophagus, stomach, small intestine, or colon) or skin. A total of 129 eligible patients were randomized to one of two treatment groups.

### Eligibility Criteria

To be eligible to enter the study, patients of any race, age, or gender met the following inclusion criteria:
1. Receipt of an allogeneic hematopoietic cell transplant ≧10 days prior to screening.
2. Symptoms consistent with Grade II intestinal GVHD with endoscopic evidence of Grade II GVHD without another plausible etiology (See Table 1).
3. Diagnosis of GVHD confirmed by biopsy of the intestine (esophagus, stomach, small intestine, or colon) or skin (histologic diagnosis within 72 hours prior to the first dose of study drug). Note: if the physician deems that it is inadvisable to do an intestinal biopsy, justification should be stated in the Case Report Form (CRF).
4. Confirmed absence of intestinal infection within 7 days prior to the first dose of study drug.
5. Demonstrated ability to swallow two tablets of the size and configuration of study drug.
6. Anti-candidal prophylaxis of the oropharynx with an effective drug prior to the first dose of study drug.
7. If female and of childbearing potential, must be willing to use adequate contraception, as determined by the investigator, for the duration of the study.
8. Ability to read, understand, and sign (or has legal representative sign) appropriate patient informed consent or assent form.

To be eligible to enter the study, patients did not meet any of the following exclusion criteria:
1. Skin GVHD, other than a slowly evolving rash that involves ≦50% of the body surface.
2. Liver GVHD with serum bilirubin >3 mg/dL.
3. >1000 mL/day diarrhea on any 1 day within 3 days prior to first dose of study drug.
4. Negative intestinal biopsy for GVHD.
5. Systemic (oral or parenteral) prescription corticosteroid use for the purpose of prophylaxis or treatment of GVHD or another inflammatory disease process within 30 days prior to first dose of study drug. (Note: Patients may continue GVHD prophylaxis with immunosuppressants including cyclosporin, tacrolimus, sirolimus, methotrexate, and mycophenolate mofetil. Use of corticosteroids such as Decadron as an anti-emetic during conditioning therapy, or use of single doses of corticosteroid in conjunction with infusion of blood products or medications to lessen side effects of these infusions, will not exclude patients from study).
6. Persistent vomiting of oral intake that precludes ingestion of study drug tablets.
7. Multi-organ failure, sepsis syndrome, or other condition with high mortality (i.e., life expectancy less than 3 months).
8. Infection of the mouth or esophagus with a fungal organism.
9. Known HIV seropositivity.
10. Pregnancy or lactation.
11. Previous use of BDP tablets, capsules, or inhalation products.
12. Use of any investigational drug, biologic, or device within 30 days prior to first dose of study drug.
13. Inability to comply with the study procedures and scheduled study visits.

### Study Drug

Beclomethasone 17, 21-dipropionate (BDP) is a diester of beclomethasone, a halogenated corticosteroid. For this study, it was supplied and administered in both immediate release (IR) and enteric coated (EC) tablets, both of which consist of the same core formulation containing 1 mg BDP per tablet. Placebo IR and EC tablets were identical in all respects to the BDP IR and EC tablets except for the absence of BDP.

The compositions of the 1 mg IR tablet and of the 1 mg EC tablet are shown in Table 2. The total daily dose was equally divided between the two forms. The rationale for using both dosage forms was to deliver BDP to both the proximal and distal portions of the gastrointestinal tract, with the objective of maximizing the efficacy in treating gastrointestinal inflammation.

TABLE 2
Composition of 1 mg BDP Immediate Release
Tablets and Enteric Coated Tablets

| Component | Quantity |
|---|---|
| Immediate Release Tablet and Enteric Coated Tablet | |
| Core | |
| Beclomethasone dipropionate, USP | 1.0 |
| Lactose monohydrate, NF | 152.0 |
| Microcrystalline cellulose, NF | 40.0 |
| Croscarmellose sodium, NF | 2.0 |
| Povidone, USP | 4.0 |
| Magnesium stearate, NF | 1.0 |
| Enteric Coated Tablet Coating | |
| Methylacrylic acid copolymer dispersion (Type C), NF | 11.34 |
| Triethyl citrate, NF | 1.7 |
| Polysorbate 80, NF | 0.025 |
| Silicon dioxide, NF | 0.91 |
| Sodium hydroxide, NF | 0.03 |
| Total tablet core | 200.0 |
| Total tablet coating | ~14.0 |
| Total | ~214.0 |

NF = National Formulary; USP = United States Pharmacopoeia

### Study Design

This was a multi-center, randomized, double-blind, placebo-controlled, parallel-group study in patients with Grade II GVHD with GI symptoms. To determine eligibility for entry into the study, patients must undergo a screening evaluation, including intestinal or skin biopsy and infection work-up.

One hundred twenty-nine patients were enrolled and randomized to receive oral BDP (8 mg/day) or matching placebo tablets for 50 days. The randomization was stratified by the source of the allograft (two HLA haplotype identical sibling versus all other), and by topical corticosteroid use (yes versus no). All patients received 10 days of prednisone at 1 mg/kg/day (or an equivalent dose of intravenous [IV] corticosteroid). On Study Day 10, patients determined by the investigator to have their GVHD controlled had their prednisone tapered rapidly over 7 days starting on Study Day 11, and received a maintenance prednisone dose of 0.0625 mg/kg/day for the remainder of the study. BDP doses were evenly divided to include equal quantities of the IR and EC formulations administered four times per day.

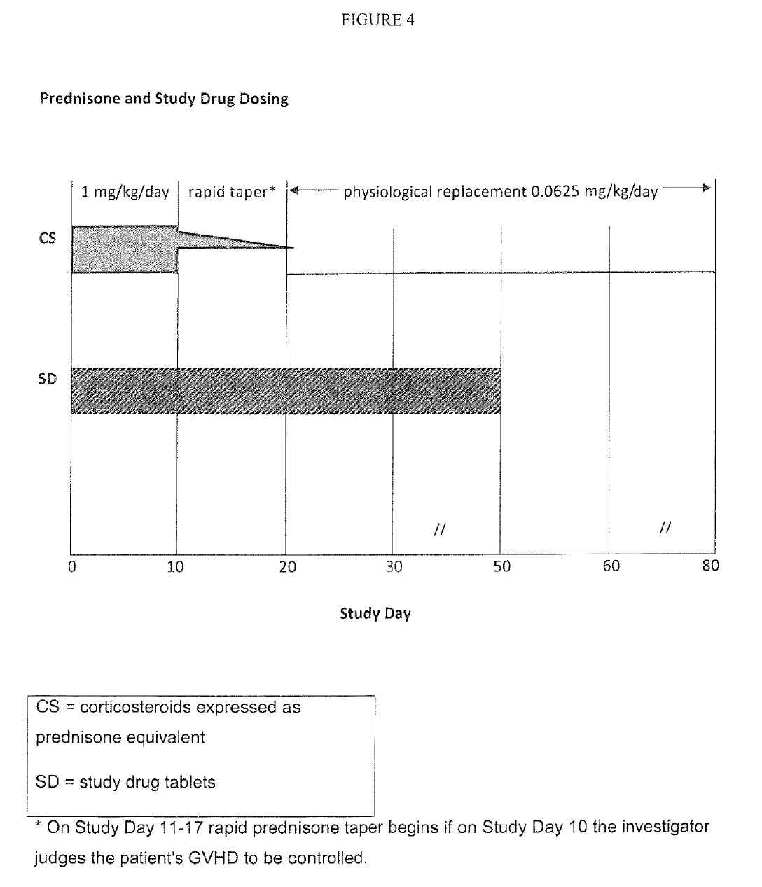

Prednisone and Study Drug Dosing is shown in FIG. 4. On Study Days 11-17, rapid prednisone tapering began if on Study Day 10, the investigator judges the patient's GVHD to be controlled.

Screening/baseline evaluations were performed within 3 days prior to the first dose of study drug, except for the confirmed absence of intestinal infection, which may be determined within 7 days. All randomized patients began receiving study drug the morning of Study Day 1, after which some patients will have timed blood samples obtained for PK analysis for up to 24 hours after this dose. For all patients, the second dose of study drug was administered on the morning of Study Day 2, after which dosing was 4 times daily until Study Day 50.

All patients are monitored per standard post transplant routine in the hospital or outpatient clinic. Patients underwent study evaluations, included blood specimens for PK analysis, on Study Days 10, 30, 50, 60, and 80. In addition, adverse events that occurred during the interval since the prior visit are assessed and recorded. Following the final dose of study drug on the morning of Study Day 50, timed blood samples were obtained for up to 24 hours for PK analysis from some patients. All patients who had normal adrenal responsiveness at Baseline and who reached Study Day 50 without experiencing treatment failure or discontinuing study drug for other reasons returned on Study Day 51 at approximately 8 am for HPA axis function evaluation.

Patients continued their physiological replacement doses of prednisone through Study Day 80. All patients underwent clinic based study evaluations on Study Day 60 and Study Day 80. The Study Day 80 evaluation was conducted by the patient's local physician if the patient had returned home from the transplant center. In the event that a patient experienced significant medical complications and was unlikely to survive the illness, or was entered into hospice care, study drug was discontinued, and the patient did not undergo any further protocol defined study procedures or assessments.

### Study Endpoints

The primary efficacy, safety, and pharmacokinetic endpoints were as follows:

### Efficacy:

1. Treatment failure, defined as use of prednisone or equivalent IV corticosteroids at doses higher than stated in protocol, or addition of other immunosuppressant medications.
2. Karnofsky Scale score if the patient is 16 years of age or older or the Lansky Scale score if the patient is younger than 16 years of age.

### Safety:

1. Adverse events.
2. Cumulative prednisone dose (per kg of body weight).
3. Worsening of GVHD as determined through the assessment of diarrhea, rash, and bilirubin levels.
4. HPA axis function.
5. Hematology measurements (with differential, including eosinophils) at Baseline and Study Day 50.
6. Serum chemistry and urinalysis measurements at Baseline and Study Day 50.
7. Patient survival through day 200 post-transplant.

### Pharmacokinetics:

1. Plasma concentrations of BDP and its metabolites, beclomethasone 17-monopropionate (17-BMP) and beclomethasone (BOH) at serial time points following the first and last doses of BDP (Study Days 1 and 50).
2. Trough concentrations of BDP, 17-BMP, and BOH plasma concentrations on Study Days 1, 10, 30, and 50.
3. Maximum plasma concentration (Cₘₐₓ), time to Cₘₐₓ (Tₘₐₓ), area under the curve (AUC), and elimination half-life (t_{1/2}) of BDP, 17-BMP, and BOH after the first (Study Day 1) and last (Study Day 50) doses.

### Statistical Plan

### Efficacy Analyses:

The primary efficacy endpoint of this two-arm study is the time to treatment failure. The primary efficacy analysis will compare the time to treatment failure through Study Day 50 using a log-rank test controlling for patient randomization stratum. Product-limit estimates for time to failure will be provided for descriptive purposes. The null hypothesis for the log-rank test is that time to failure is homogeneous across the treatment groups.

Secondary analyses of treatment failure compared the treatment groups with respect to proportion of treatment failures and Karnofsky Scale (or Lansky Scale) scores at key time points. The difference in proportion of treatment failures on Study Days 10, 30, 50, 60, and 80 was evaluated using a two-sided Fisher's Exact test of the null hypothesis of no difference between the treatment groups. The difference in Karnofsky Scale (or Lansky Scale) scores at the same time points will be evaluated using a two-sided Wilcoxon test to evaluate the null hypothesis of no difference in medians between treatment groups.

### Safety Analyses:

Treatment-emergent adverse events were summarized by incidence, incidence within body system, incidence by severity, relatedness to study drug, and outcome. Study drug discontinuation due to adverse event(s) was summarized overall and by days on study.

The ability of BDP to spare GVHD patients the systemic corticosteroid exposure was assessed through the evaluation of cumulative prednisone dose in mg/kg over the course of the study. Descriptive statistics will be used to compare treatment groups.

The worsening of GVHD was evaluated through the assessment of diarrhea (GI), rash (skin), and bilirubin (liver) on Study Days 10, 30, 50, and 60. These endpoints will be analyzed using descriptive statistics.

The effect of oral BDP on HPA axis function was assessed by measuring plasma concentrations of adrenocorticotrophic hormone (ACTH), resting morning cortisol, and change in plasma cortisol concentration following a standard test dose of intravenous ACTH. All patients who are enrolled in the study had their HPA axis function assessed at Baseline (within 2 days prior to Study Day 0). All patients who had normal adrenal responsiveness at Baseline and who reach Study Day 50 without experiencing treatment failure were evaluated for HPA axis suppression on Study Day 51. These patients were on physiologic replacement doses of prednisone, per protocol. A patient was considered to have evidence of abnormal HPA axis function if any of the three tests described above were outside the normal range. The results between and within treatment groups were analyzed using descriptive statistics.

### Pharmacokinetic Analyses:

BDP, 17-BDP, and BOH plasma concentrations will be measured 12 times in 24 hours on Study Days 1 and 50. If the prevalence and magnitude of plasma concentrations of BDP, 17-BMP, and BOH permitted, then non-compartmental estimates of AUC, Cₘₐₓ, Tₘₐₓ, and t_{1/2} were computed for each patient randomized to BDP. Paired t-tests were used to compare the PK parameters on Study Day 1 and 50. Trough concentrations on Study Days 1, 10, 30, and 50 were summarized using descriptive statistics.

### Randomization, Study Drug Treatment Groups, and Prednisone Dosing

Patients were randomized to one of two treatment groups: 8 mg of BDP daily (administered as one 1 mg IR tablet and one 1 mg ER tablet q.i.d.) or placebo tablets. Both regimens included oral prednisone (1 mg/kg/day) or an equivalent dose of IV methylprednisolone (dose of IV methylprednisolone=0.8×dose of oral prednisone) given in 2 divided doses per day on Study Days 1 to 10.

Eligible patients fit into one of four strata: 1) two HLA haplotype identical sibling with topical corticosteroid use at baseline; 2) other allograft source with topical steroid use at baseline; 3) two HLA haplotype identical sibling graft with no topical corticosteroid use at baseline; or 4) other allograft source with no topical steroid use at baseline. After determining the correct stratum for a patient, the pharmacist assigned the next patient number in that stratum to the patient. The pharmacist dispensed the study drug according to a block randomization schedule unique to each site that is provided by Enteron.

On Study Day 10, patients were evaluated for the signs and symptoms of GVHD. Patients in both treatment groups who met the response criterion of adequately controlled GVHD, as judged by the investigator, had their prednisone tapered over 7 days starting on Study Day 11 (Table 3). Starting on Study Day 17, physiologic replacement doses of prednisone were given for the remainder of the study. If the investigator determined that GVHD was not controlled on Study Day 10, patients discontinued study drug but continued prednisone initially at 1 mg/kg/day; these patients were considered to be treatment failures. In treatment failure cases, physicians subsequently modified prednisone dosage as deemed appropriate.

TABLE 3
Prednisone Dose and Taper Schedule

| | | |
|---|---|---|
| | Prednisone | Total Daily |
| | Dosing | Prednisone |
| Study Days | Schedule* | Dose* |

| | | | | |
|---|---|---|---|---|
| 1-10 | 0.5 | mg/kg b.i.d. | 1.0 | mg/kg |
| 11-12 | 0.25 | mg/kg b.i.d. | 0.5 | mg/kg |
| 13-14 | 0.125 | mg/kg b.i.d. | 0.25 | mg/kg |
| 15-16 | 0.0625 | mg/kg b.i.d. | 0.125 | mg/kg |
| 17-80 | 0.0625 | mg/kg q.d. | 0.0625 | mg/kg |

*Or an equivalent dose of IV corticosteroid (dose of IV methylprednisolone = 0.8 × dose of oral prednisone)

Prednisone dosing calculations throughout the study were based on the weight of the patient at the Baseline Visit (Study Day 0). The Daily Diary was used to collect daily prednisone dosing information.

If signs or symptoms of adrenal insufficiency occurred during tapering, prednisone was resumed at higher doses, depending on the level of medical stress, and a more gradual taper implemented. If a patient's GVHD worsened during the tapering of prednisone, higher doses of prednisone were resumed at the discretion of the investigator, and the patient was considered a treatment failure. However, an exception was made to this definition of treatment failure if the higher dose of corticosteroids was prescribed for <96 hours to cover the possibility of adrenal hyporesponsiveness during an anticipated period of medical stress, such as surgery.

### Results

Intestinal graft-versus-host disease (iGVHD) is a life threatening condition that is one of the most common causes for the failure of bone marrow transplant procedures. These procedures are being increasingly utilized to treat leukemia and other cancer patients with the prospect of eliminating residual disease and reducing the likelihood of relapse. BDP represents a first-of-its-kind oral, locally acting therapy tailored to treat the intestinal manifestation of GVHD, the organ system where GVHD is most frequently encountered and highly problematic. BDP is intended to reduce the need for systemic immunosuppressives to treat iGVHD. Currently approved systemic immunosuppressives utilized to control iGVHD substantially inhibit the highly desirable graft-versus-leukemia (GVL) effect of bone marrow transplants, leading to high rates of aggressive forms of relapse, as well as substantial rates of mortality due to opportunistic infection.

### Study Design

One hundred and twenty-nine (129) post-bone marrow transplant patients presenting Grade II iGVHD were enrolled in the randomized, double-blind, placebo-controlled, multi-center clinical trial, which was conducted at 16 bone marrow transplant centers in the United States and France. All patients in the Phase III clinical trial were initially treated with a constant daily dose of high dose (1-2 mg/kg) prednisone, which is the current standard therapy, in combination with an oral dose of either BDP (8 mg/day) or placebo for the first 10 days. On day 10, if patients were responding to treatment, the high dose prednisone was rapidly tapered and patients continued to receive either BDP or placebo orally for an additional forty days. The primary endpoint of the study was a comparison between the two treatment arms of the time to treatment failure, defined as the need for additional therapies due to uncontrolled signs or symptoms of GVHD. Secondary endpoints in the study included a comparison of the proportion of treatment failures and clinical scores at 10 Days, 30 Days, 50 Days, 60 Days, and 80 Days post-randomization, a comparison of cumulative exposure to systemic steroids, as well as a comparison of mortality at Day 200 post-transplant.

Current Top-Line Data

| | | | p-value |
|---|---|---|---|
| | orBec ® | Placebo | orBec ® v. |
| Endpoints | n = 62 | n = 67 | Placebo |
| Time to Treatment Failure at | n/a | n/a | 0.1177 |
| Study Day 50 (primary endpoint) | | | |
| Treatment Failure Rate at Study | 31% | 48% | 0.0515 |
| Day 50 | | | |
| Time to Treatment Failure at | n/a | 52 | 0.0226 |
| Study Day 80 | | | |
| Treatment Failure Rate at Study | 39% | 65% | 0.0048 |
| Day 80 | | | |
| Mortality Rate at 200 Days Post- | 5 (8%) | 17 (26%) | 0.006 |
| Transplant | | | |

n/a = not achieved due to statistical methodology

### Time to Treatment Failure

While BDP did not achieve statistical significance in its primary endpoint of time to treatment failure through Day 50 (p-value 0.1177), BDP did achieve statistical significance in its secondary endpoint of time to treatment failure through Day 80 (p-value 0.0226). The Company believes that the p-value of 0.1177 achieved in the primary endpoint through Day 50 is primarily due to a higher than expected rate of treatment failures during Days 0-10 of the study. During such period, patients were receiving high dose prednisone (1-2 mg/kg/day) plus either BDP (8 mg/day) or placebo.

For purposes of the study, patients that did not begin the rapid taper of high dose prednisone on Day 10 as called for by the regimen were deemed treatment failures for all purposes, including the calculation of statistical significance of time to treatment failure at Day 50. The Company intends to further analyze the Days 0-10 treatment failure group and the statistical impact of this group on the primary endpoint of time to treatment failure at Day 50 and discuss the results of this analysis with the FDA. Encouragingly, the treatment failure rate at Day 50 approached statistical significance (p-value 0.0515). In addition, the secondary endpoint of time to treatment failure at Day 80, as well the treatment failure rate at Day 80, each achieved statistical significance (p-values 0.0226 and 0.0048, respectively).

orBec® Demonstrates Highly Statistically Significant Reduction in Mortality

Perhaps of greatest clinical relevance, orBec® demonstrated a 70% reduction in mortality, registering only 5 (8%) deaths during the prospectively defined Day 200 post-transplant period versus 17 (26%) deaths for the placebo group (p-value 0.006). Based upon separate analysis conducted by the Company, there is also a statistically significant correlation between treatment failure and mortality.

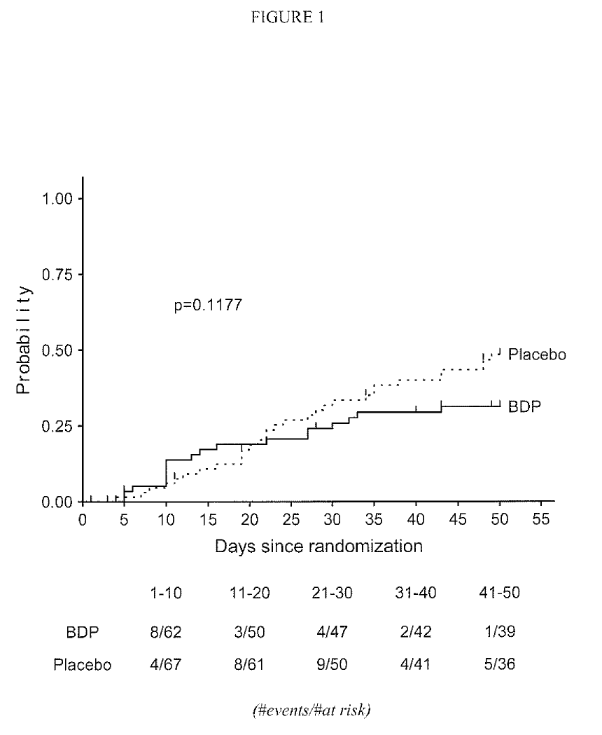

FIG. 1 shows the cumulative treatment failures during the first 50 days in patients receiving placebo or BDP.

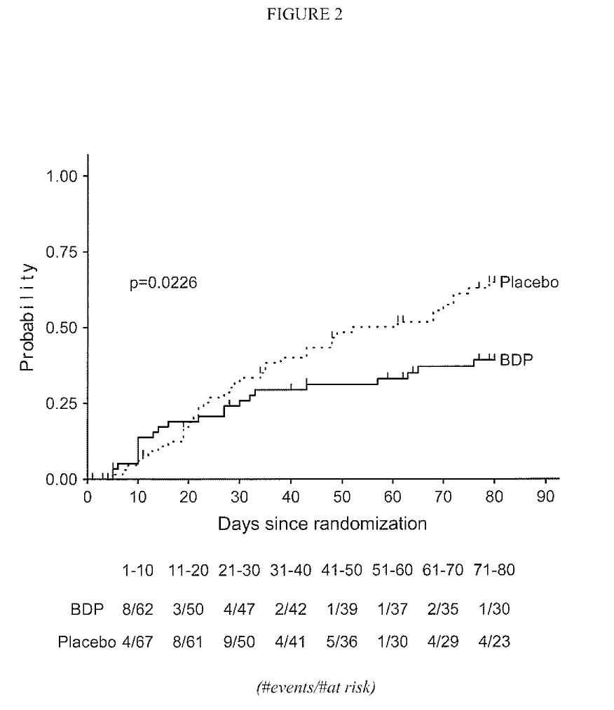

FIG. 2 show the cumulative treatment failures during the first 80 days in patients receiving placebo or BDP.

TABLE 4
Cumulative Treatment Failure Rate at Specified Time Points
Estimates Based on Kaplan-Meier Method (Full Analysis Set)

| | Placebo | BDP |
|---|---|---|
| Subjects randomized | 67 | 62 |
| Study Day 10 | | |
| Cumulative events | 4 | 8 |
| Treatment failure rate (95% | 0.06 (0.03, 0.13) | 0.14 (0.08, 0.24) |
| CI) | Z = 1.426, P = 0.1538 | |
| Study Day 30 | | |
| Cumulative events | 21 | 15 |
| Treatment failure rate (95% | 0.33 (0.25, 0.45) | 0.25 (0.18, 0.37) |
| CI) | Z = 0.911, P = 0.3625 | |
| Study Day 50 | | |
| Cumulative events | 30 | 18 |
| Treatment failure rate (95% | 0.48 (0.39, 0.60) | 0.31 (0.23, 0.43) |
| CI) | Z = 1.948, P = 0.0515 | |
| Study Day 60 | | |
| Cumulative events | 31 | 19 |
| Treatment failure rate (95% | 0.50 (0.41, 0.62) | 0.33 (0.24, 0.45) |
| CI) | Z = 1.913, P = 0.0558 | |
| Study Day 80 | | |
| Cumulative events | 39 | 22 |
| Treatment failure rate (95% | 0.65 (0.55, 0.76) | 0.39 (0.30, 0.52) |
| CI) | Z = 2.819, P = 0.0048 | |

The cumulative treatment failure rate is estimated from the complement of the Kaplan-Meier distribution.
The variance (standard error) of the Kaplan-Meier estimate is calculated using Greenwood's formula.
Test of difference in treatment failure rates (Placebo − BDP) based on the Z-test.
Significance level of 0.05 (two-sided). No adjustments for multiple tests of significance.

TABLE 5
Overall Survival Status - 200 Days
Post-Transplant (Safety Population)

| | Placebo | | BDP | | Overall | |
|---|---|---|---|---|---|---|
| Subjects | 67 | | 62 | | 129 | |
| randomized | | | | | | |
| Subjects | 66 | | 61 | | 127 | |
| evaluable | | | | | | |
| for safety* | | | | | | |
| Survival status | | | | | | |
| Alive | 49 | 74% | 56 | 92% | 105 | 83% |
| Dead | 17 | 26% | 5 | 8% | 22 | 17% |

| | | | |
|---|---|---|---|
| Day 200 | 0.74 (0.66, 0.84) | 0.91 (0.86, 0.98) | 0.83 (0.77, 0.88) |

| | |
|---|---|
| survival | Z = 2.750, P = 0.0060 |
| rate (95% CI) | |

We claim:

1. A method of treating a patient requiring long term therapy following hematopoietic cell transplantation having graft-versus-host disease or following organ allograft transplantation having host-versus-graft disease, the method comprising long term topical oral administration of beclomethasone wherein treatment is directed to tissue selected from the group consisting of intestine and liver and further wherein the beclomethasone dipropionate is initially administered beginning 29 days post transplantation through 56 days post transplantation.

2. The method of claim 1 wherein the beclomethasone dipropionate is administered orally at a dosage of 4 mg per day to 12 mg per day.

3. The method of claim 1 wherein the patient has tissue damage and the tissue is intestinal mucosa.

4. The method of claim 1 wherein the patient has tissue damage and the tissue is small bile ducts of the liver.

5. The method of claim 1 wherein the patient has tissue damage and the tissue is inflammation.

6. The method of claim 1 wherein the patient has tissue damage and the tissue damage is destruction of mucosa of the intestine.

7. The method of claim 1 wherein the beclomethasone dipropionate is administered orally from day 29 to day 56 following hematopoietic cell transplantation.

8. The method of claim 1 wherein the beclomethasone dipropionate is administered in combination with predisone and prednisolone at 2 mg per kg.

9. The method of claim 1 wherein the beclomethasone dipropionate is formulated for oral administration in the form of a pill, capsule or microsphere.

10. The method of claim 1 wherein the beclomethasone dipropionate is formulated such that the pill, microsphere, or capsule dissolves in the stomach, small intestine or colon.

11. The method of claim 1 wherein the beclomethasone dipropionate is formulated for oral administration in the form of an emulsion.

12. The method of claim 1 wherein administration of the beclomethasone dipropionate initiates following infusion of the hematopoietic cells.

13. The method of claim 1 wherein administration of the beclomethasone dipropionate ceases after 80 days following infusion of the hematopoietic cells.

14. The method of claim 1 wherein the patient is the recipient of HLA-mismatched hematopoietic stem cells.

15. The method of claim 1 wherein the patient is the recipient of unrelated donor hematopoietic stem cells, umbilical vein hematopoietic stem cells, or peripheral blood stem cells.

16. The method of claim 1 wherein the beclomethasone dipropionate is administered in combination with other prophylactic agents.
